(19) United States
(12) Patent Application Publication
(10) Pub. No.: US 2006/0190073 A1
(43) Pub. Date: Aug. 24, 2006

(54) Method and Apparatus for Making a Braided Stent with Spherically Ended Wires

(75) Inventors: Gary Nachreiner, Mound, MN (US); Robert L. Rykhus JR., Edina, MN (US); Sidney Hauschild, Brooklyn Park, MN (US); Mark Polyak, Minnetonka, MN (US)

Correspondence Address: AMS RESEARCH CORPORATION, 10700 BREN ROAD WEST, MINNETONKA, MN, 55343, US

(73) Assignee: AMS Research Corporation

(21) Appl. No.: 11/379,947

(22) Filed: Apr. 24, 2006

Related U.S. Application Data
(62) Division of application No. 09/749,291, filed on Dec. 27, 2000.

(51) Int. Cl. A61F 2/06

(52) U.S. Cl. 623/1.15

(57) ABSTRACT

A method and apparatus for cutting a braided wire stent to a predetermined length such that a ball or sphere is formed on the end of each cut wire of the stent. These spheres are advantageous in that they provide added comfort to the patient and also act against the other wires of the stent to prevent the stent from becoming unbraided during the process of collapsing and expanding the stent such as is done when the stent is being inserted into a patient. The apparatus releasably holds and precisely positions the wires while the spheres are being formed.

### CLAIM FOR PRIORITY

This divisional patent application claims priority to United States utility patent application Ser. No. 09/749,291, filed Dec. 27, 2000, and entitled “Method and Apparatus for Making a Braided Stent with Spherically Ended Wires.” The identified utility patent application is herein incorporated by reference.

### TECHNICAL FIELD

The present invention pertains generally to cutting braided stents from stock.

### BACKGROUND OF THE INVENTION

Stents are generally metal or plastic tubes inserted into a vessel such as the urethra to keep a lumen open. A vast variety of stent materials and designs are available. A few examples of available designs include braided tubes, wire springs, and tubes having a plurality of holes formed therein to provide flexibility. It is preferable that a stent design provides a tube which can be stretched or otherwise manipulated to reduce the diameter of the tube while the stent is being inserted, and which expands to resume an original outside diameter when released. Reducing the diameter of the stent during insertion reduces the likelihood of trauma to the surrounding tissue of the lumen into which the stent is being inserted. Of the available designs, a stent braided from thin wires is particularly suited for this purpose in that, when stretched, its diameter is rapidly reduced relative to the measure of stent elongation. Furthermore, the energy stored in the stent when the stent is stretched is relatively small, so that when the stent returns to its original shape within the lumen, it does so at a safe rate in a gentle manner without damaging the surrounding tissue. Conversely, in order to reduce the diameter of a coiled spring, the spring must either be pulled, creating spaces between the coils of the spring which may potentially provide a pinch hazard, or twisted several times, setting up a potentially significant recoil force which may impart damage to soft tissue when released.

Braided stents, however, have posed certain problems pertaining to their manufacture and use. The stents are cut from a length of braided tubular stent stock. The stock typically comprises a plurality of right-handed helical wires or strands interwoven with an equal number of left-handed helical wires or strands. Each wire or strand has a first end and a second end. The first ends of all the strands together generally define the first end of the stock and the second ends together generally define the second end of the stock. All of the wires or strands form helixes that have substantially equal outside diameters, twist angles, and share a common central axis. Ideally, all of the right-handed helixes are angularly spaced apart from each other by an equal angle, as are the left-handed helixes. This creates a diamond pattern formed by the intersecting strands wherein the intersections form the apexes of the diamonds and the individual strands between the intersections form the sides of the diamonds. Equally spaced apart helixes ensure that the diamond pattern further forms uniform rows of adjacent diamonds arranged so that the upper and lower apexes are substantially aligned and the side apexes are also aligned. Ideally, a line connecting the upper and lower apexes should be perpendicular to a line connecting the side apexes. The interwoven helical strands together generally define a stent periphery which is generally cylindrical.

Some of the problems presented by using braided stock to form stents arise when inconsistencies are found in the individual diamond dimensions. When the angular spaces between the individual helixes are not uniform, the apexes quickly become misaligned. Attempts at cutting such a stent along a plane that is substantially perpendicular to the central axis of the stock results in free wire ends of varying lengths and angles. Moreover, devising an automated method or mechanism for cutting a braided stent is significantly complicated by pattern irregularities.

For example, stent stock may be placed on a mandrel for automatic cutting by a device which provides a cutting force, whether it be heat or a mechanical force. The mandrel carrying the stock rotates around its central axis while the cutting force cuts each individual wire as they pass beneath the cutting device. This results in a cutting plane that is perpendicular to the axis of rotation. If the stent stock has an irregular diamond pattern, the cuts will occur at various positions between apexes or at the apexes themselves. This is undesirable for several reasons. The spaces between the wire ends will vary and may increase the discomfort experienced by the patient. Also, the tendency for the stent to become unraveled is significantly increased due to the varying lengths of strand portions that extend beyond the apexes adjacent the cutting plane. Additionally, the ability of the stent to be compressed and released is degraded due to the increased tendency of the stent to unravel as the wires slide relative to each other when the stent is compressed and released. If heat is used to cut the stent, and the heat source gets too close to the intersections of the strands, the adjacent strands forming the intersection may become welded together, inhibiting the ability of the braided stent to be compressed without becoming deformed.

Other problems presented by braided stents pertain to the ends of the individual wires. Once the wires are cut, they tend to provide sharp edges. These edges may irritate the walls of the lumen or vessel in which the stent is being used, thereby causing discomfort to the patient, and may make removal of the stent more difficult, should removal be necessary. Additionally, the sharp edges provide little to no resistance to the unraveling problem mentioned above.

Attempts at developing an automated manufacturing method, which overcomes these problems, have failed. For example, in order to present a uniform diamond pattern to the cutting device, efforts have been made to manipulate the diamond pattern by moving the individual wires into a desired formation. One effort incorporated a mandrel with helical grooves cut into the outer surface for receiving the braided stent therein. Unfortunately, these procrustean efforts resulted in creating internal stresses in the wires. Once the wires were cut, the stresses were released, and the wires “jumped” apart. This jumping action not only created additional unraveling problems, it frustrated attempts at shaping the resulting wire ends to provide a dull surface because the wire ends jumped out of operable proximity with the cutting device.

Methods including visual wire location means have also been attempted with unsatisfactory results. Locating wires visually avoids some of the manipulation issues described above, but can be labor intensive and time consuming. Moreover, the stents produced contain inconsistencies due to operator inaccuracies inherent in the visual location methods.

Shaping the wire ends to provide a dull surface may reduce the discomfort presented to the patient by sharp wire ends. Methods have been developed which form spheres on the ends of wires. These spheres are desirable because they provide a dull surface and, more importantly, because the resulting spheres generally have a diameter greater than that of the wire. This increased diameter effectively reduces the tendency of the braided stents to become unraveled. When a braided stent is stretched or compressed, the individual helical wires or strands slide relative to each other. As they slide, the positions of the intersections move relative to the wire ends. If the location of the intersection moves to the ends of the wires, there is a tendency for the wires to unravel and attempt to achieve a straighter shape. Providing spheres at the ends of the wires or strands reduces this tendency by presenting a physical barrier to wire ends passing over wires with which they intersect, thereby preventing unraveling.

Unfortunately, attempts at developing an automated manufacturing process to create these spheres have heretofore been unsuccessful. Some of the reasons pertain to the inconsistencies in the braided diamond patterns, others pertain to the alternating angles presented by the interwoven helixes. Explanation of these reasons requires a brief discussion of sphere formation.

It has been found that melting the ends of the strands can result in such a sphere when a focused heat source is directed to a point on the wire and then moved along a predetermined length of the wire toward the desired location of the sphere. Doing so causes molten strand material to follow the wire ahead of the heat source, accumulating to form a sphere.

If a strand of meltable material, such as metal or plastic, passes through a heat source, a section of the strand will be melted away to form a gap in the strand, provided the heat source is hot enough to melt the material. The length of this gap, measured in a direction perpendicular to the direction of relative movement of the heat source, will define the effective cutting width of the heat source. The effective cutting width may be increased by providing a larger heat source, or by making multiple passes with the same heat source and laterally offsetting the path of the heat source on each subsequent pass.

When a strand of meltable material under stress, such as the stress found in a wire which has been braided into a helix, is subjected to such a heat source, the molecular bonds being stretched by the stress will break and the strand will separate as the stress is relieved. Depending on the amount of tension in the strand, the newly formed ends of the strand, defining the gap, may remain subjected to the heat source and will melt and tend to move away from the heat source by following the adjacent solid portions of the strands. When the liquid cools and solidifies on the strand, the thickness of the strand is increased. This phenomenon is due to the surface tension of the liquid formed when the material melts. Surface tension causes a drop of liquid to minimize its surface area. Therefore, a drop of liquid having surface tension tends to attach itself to a solid rather than dropping off. This tendency occurs because a drop of fluid on a solid has a smaller overall surface area than a suspended drop. Similarly, surface tension also causes a body of liquid to form a sphere when the body is not acted upon by any other external forces. A sphere, geometrically, has the smallest surface area of any shape per unit volume.

The magnitude of the increase in thickness will vary with the amount of liquefied material collected on the end of the strand, and, when the body is under the influence of gravity, by the strength of the surface tension relative to the weight of the material. The increase in thickness will also vary depending on the amount of heat absorbed by the liquid. The surface tension of a liquid is inversely proportional to its thermal energy. In other words, liquids become thicker as their temperatures approach freezing.

If the strand is oriented such that its direction of travel is substantially perpendicular to its longitudinal axis, as the strand passes in operational proximity to the heat source, the strand will separate, as discussed above, and the newly formed ends defining the gap will spend relatively little time exposed to the heat source. The result will be insignificant increases in thickness on both newly formed ends. In order to form a significant sphere on one end, the wire is preferably oriented to approach the heat source such that an acclivitous angle is formed between the path of the wire and its longitudinal axis, with the sphere usually resulting at the top of the slope. Alternatively, the wire may be fed into the heat source along its longitudinal axis, but the heat source must be turned off when the sphere has achieved a desired size. It will become apparent that this path is not conducive to automating the process of forming spheres on the ends of the wires of braided stent stock.

It is to be understood by those skilled in the art that movement between the heat source and the wire is relative. Whether the heat source is physically moved toward the wire or the wire is physically moved toward the heat source, or any combination thereof, is inconsequential for purposes of the discussion herein or when practicing the teachings of the invention. For ease of explanation of FIG. 1, the heat source will be described below as moving toward a wire or strand.

FIG. 1 presents a series of sequential diagrams showing the formation of a sphere S as a focused heat source passes through a wire at an acclivitous or upwardly sloping angle. Due to the relative acclivitous angle δ between the path P, having a width w of heat source H and the wire 14, heat source H first makes contact with wire 14 near the bottom of heat source H. Once contact is made, heat source H cuts wire 14 into two pieces, thereby creating a bottom end B and an upper end U. As heat source H continues along path P, it continues to melt upper end U and moves past bottom end B rather quickly. It can be seen that, when wire 14 is presented at an acclivitous angle 6 to heat source path P, a sphere S forms above heat source H as heat source H continues to collide with and move through wire 14. A significant sphere S does not form on wire 14 below heat source H because the bottom end B of the wire 14 loses contact with heat source H after the initial cut and therefore, little to no strand material accumulates on end B.

It should be noted that the cutting effect is due, in part, to the tension in the wire 14, as described above. Notably, if the tension is too great, the wire 14 will spring apart quickly and take the bottom end B and the upper end U out of operably proximity with heat source H so that spheres S are not formed. Conversely, if there is little or no tension in wire 14, the wire may not separate immediately and both upper end U and bottom end B will remain within operable proximity to the heat source H long enough to form spheres S on both ends.

The size of the formed sphere S is dependent on the size of the wire 14 and the amount of energy delivered to the wire. The amount of energy delivered to the wire is dependent on the temperature of the heat source H and the amount of time the wire 14 spends in operable contact with the heat source H. The amount of time the wire 14 spends in operably contact with the heat source H may be controlled by varying the relative speed between the heat source H and the wire 14, and is dependent on the angle 6 presented between the wire 14 and the path of the heat source H.

If the relative speed between the heat source H and the wire 14 is too fast, the wire 14 may not absorb enough heat to melt and separate or the wire 14 may separate but the amount of material melted by the heat source may be too small to a form significant sphere S. If the relative speed is created by rotating the stent around a central axis in operable proximity to a stationary heat source H, excessive angular velocity may result in a sphere S becoming radially displaced outwardly from the centerline of the wire 14 due to centrifugal force. A stent with wire ends having such radially displaced spheres S will have an increased maximum outer diameter which may provide increased discomfort and insertion and removal difficulties.

If the angle δ presented is too shallow, the relative speed between the heat source H and the wire 14 must be slower because the component of the relative speed in the direction of path P will be greater. Also, sphere S will end up being larger because more wire material will be lying in path P. This may result in the loss of sphere S due to the inability of the surface tension to overcome the forces of gravity. In short, sphere S may drip off of wire 14 before it escapes path P and has a chance to cool on wire 14. Conversely, if the angle δ is too steep, there will be insufficient wire material to form a significant sphere S.

Predictably, attempts at forming a stent of braided strands with spherical ends using an automated process have struggled with presenting each wire at an appropriate angle to the heat source, ensuring that the heat source path intersects the wire between the apexes, providing an appropriate relative speed between the wire and the heat source, and manipulating the stent stock without creating internal stresses within the wire so that the wire doesn't “jump” out of the path of the heat source when initially cut. Additionally, braided stents, being formed of alternating left-handed and right-handed helixes, present alternating acclivitous and declivitous angles to a heat source travelling relative to a rotating stent. Cutting each wire in sequence would result in spheres formed on alternating sides of the cut.

It can be seen that there is a need for an automated method of cutting a stent from a length of braided stock material.

There is also a need for an automated method of cutting a stent from a length of braided stock material that overcomes some or all of the problems described above.

More specifically, there is a need for a device for holding a length of braided stock material that does not allow the individual wire to “jump” after being cut.

There is also a need for an automated method of cutting a stent from a length of braided stock material that results in a uniform plurality of wire ends.

There is, more specifically, a need for an automated method of cutting a stent from a length of braided stock material that incorporates a typical laser cutting machine having a laser and an axially displaceable indexing head.

There is yet a further need for an automated method of cutting a stent from a length of braided stock material that creates a sphere or similar dull surface at the end of each wire of the stent.

There is an additional need for a method of cutting a stent from a length of braided stock material that results in a braided stent that is resistant to unraveling.

There is a further need for a method of cutting a stent from a length of braided stock that creates a stent that provides increased comfort to the patient.

### SUMMARY OF THE INVENTION

The present invention pertains generally to an automated method and apparatus for cutting a stent having a predetermined length from a length of braided stent stock.

In a preferred form, the present invention provides a device that temporarily secures a length of stent stock so that it may be controllably moved relative to a heat source used to cut the stent. This device preferably includes an elongate mandrel having an outside diameter slightly smaller than the inside diameter of the relaxed, braided stent stock the mandrel is designed to secure. A length of stent stock is placed on the mandrel at a predetermined axial position along the length of the mandrel. The mandrel also preferably defines a central or inner channel having an inside diameter sized to receive an elongate activation dowel. An anchoring or compensating mechanism, operably attached to the mandrel, releasably fixes the stent stock to the mandrel and compensates for irregularities in the stent braiding by gently manipulating the individual wires of the stent to present the sections of the wires that are between diamond apexes, to a heat source in a predictable, repeatable manner.

A preferred embodiment uses a laser as the melting source or heat source such as that found on the Eagle 500 CO₂ Laser System, manufactured by Laser Machining, Incorporated of Somerset, Wis. A laser is advantageous because it is extremely focused and emits relatively little radiant heat. In other words, the temperature gradient, as the distance from the center of the laser increases, is very steep. Lasers can also be shuttered on and off very quickly by using shutters or deflectors to block the beam from coming into operable contact with the target. It is envisioned, however, that other, similarly focusable heat sources may be used without detracting from the spirit of the invention.

Preferably, the anchoring mechanism includes at least one set of two or more angularly spaced apart apertures extending radially through said mandrel. These apertures house inwardly biased, outwardly displaceable, pins or protuberances, constructed and arranged to slide in and out of the apertures when the activation dowel is inserted or engaged, and removed or disengaged.

The activation dowel begins at a first end, includes a handle portion and an activation portion, and concludes at a second end. The handle portion is preferably cylindrical and has an outside diameter slightly smaller than that of the inside diameter of the channel defined by the mandrel. Preferably the handle portion slides easily in and out of the mandrel, however, is snug enough to avoid any appreciable play. The activation portion includes one or more surfaces, preferably continuous surfaces, which act on the pins in sequence causing them to protrude when the activation dowel is inserted within the mandrel. The angled portion gradually increases the diameter of the dowel from an angled portion distal end to an angled portion proximal end.

It is understood that the channel and the activation dowel may be of any shape and do not necessarily have to be cylindrical. Similarly, the angle portion could be the frustum of a cone, pyramid, or any other shape of increasing or decreasing diameter. Clearly, in order to lower manufacturing costs and time, the cylindrical relationship between the dowel and the channel, herein described, is preferable.

The pin protrusion sequence caused by the angled portion is advantageous. When a mandrel provides more than one set of pins, each set being displaced from a preceding set by a predetermined longitudinal distance, the angled surface causes the first set it encounters during its insertion to protrude from the outer surface of the mandrel before the next set of pins is acted upon by the angled surface. This progressive pattern of activation is advantageous in that the first set of pins functions to generally align the braided stent stock with the pins so that the second and, preferably, third sets of pins may find the appropriate respective spaces in the stent stock more easily while engaging the stent stock. In the event that the first set of protuberances should happen to abut directly against the strands of the stent stock while they are emerging from the apertures, the stent stock may be slid slightly along the axis, either forwardly or rearwardly, in order to free the stock from the interference. Alternatively, the stent stock may be rotated slightly to expose the pins or protuberances to the spaces. Subsequent sets of protuberances or pins should then be free of any interference as they are engaging the stock.

It is envisioned that the present invention includes pins or protuberances that are sized to snugly fit within the diamond shaped holes defined by the strands of the braided stent stock. Sizing the pins thusly results in a more secure relationship between the stent stock and the mandrel and reduces the likelihood of manufacturing errors due to stock movement.

The combination of the progressive engagement pattern described above with pins sized to snugly fit within the diamond shaped holes defined by the strands of the braided stent stock ensures that the mandrel adequately compensates for irregularities in the braided stent design.

In another aspect, the present invention provides a device for securing a length of stent stock, as described above, at a predetermined axial position along the device, which includes two sets of spaced apart pins separated by a cutting groove. Providing two sets of pins, preferably including four pins per set, and a cutting groove between the sets, significantly decreases the tendency for the wires to “jump” away from the cutting tool after the cut has been made.

In other aspects of the present invention, the mandrel includes three sets of pins, preferably having at least two pins per set, more preferably three pins per set, and even more preferably four pins per set, and two cutting grooves juxtaposed between each of the sets of pins such that one set of pins lies between the cutting grooves while the remaining sets are found on the outside of each groove. This arrangement is advantageous in that it facilitates a faster manufacturing process, and provides more accurate positioning of wires and intersections relative to the position of the heat source, than does the use of fewer pins.

More specifically, the braided stent stock is placed on the mandrel so that numerous stents may be cut therefrom. Though each cut results in two ends of stock, usually only one end has spheres formed on the ends of the individual strands. Another cut must be made to form spheres on the other end of the stock. In other words, in order to cut a plurality of stents with spherically ended strands from a single length of stock, a certain amount of waste must be allocated between each strand. Providing two grooves, spaced apart by a distance which will result in the length of the scrap piece, allows the end of one stent to be cut, and the beginning of another stent to be cut, without adjusting the position of the stent stock on the mandrel. This is also advantageous in that it results in a predictable, repeatable length of scrap between each stent. In this embodiment, three sets of pins are provided so the strands of the stent stock are secure on either side of each cutting groove. This prevents each strand from “jumping” out of alignment after it is cut.

In one aspect of the present invention, springs are provided, operably attached to each pin, thereby biasing the pins toward an inward position whereby a smooth outer mandrel surface is provided when the activation dowel is not inserted. This arrangement facilitates sliding a newly formed stent and scrap pieces off of the mandrel and also allows the remaining length of stent stock to be slid along the length of the mandrel so that another stent may be cut therefrom.

In another preferred aspect of the present invention, a method of cutting a stent of a predetermined length from a length of braided stent stock is provided. This method preferably involves using a focused heat source capable of creating an area of heat sufficient heat to melt a predetermined length of one of the elongate strands of the braided stock. A length of the braided stent stock is provided and aligned with the heat source such that the heat source is aimed substantially between two adjacent rows of vertices formed by the intersections of the individual strands.

Once the heat source is properly aligned with the stent stock at the desired cutting location, the stent stock is rotated relative to the heat source around the central axis of the stock. Preferably, the heat source remains between the two adjacent rows of vertices while the stock is rotating.

It has been found that a preferable way to form predictable, consistent spheres on one side of the cut involves subjecting alternating strands to the heat source such that only strands having substantially acclivitous angles relative to the path of the heat source are melted, thereby forming a sphere on every other strand proximate the upper side of the area of heat. Once all of the strands presenting acclivitous angles relative to path of relative motion of the heat source are melted, the relative path of motion is reversed such that the remaining strands now present acclivitous angles to the path of the heat source. The remaining strands are then cut and spheres are formed proximate the upper side of the area of heat.

This preferred aspect of the present invention preferably incorporates a turning mechanism for controllably rotating the stent stock beneath the cutting device at a controlled, predetermined angular speed. This predetermined angular speed is preferably calculated to ensure proper sphere formation at the ends of the individual strands,

More preferably, the mandrel is constructed and arranged for insertion into a laser cutting machine, such as the Eagle 500 CO₂ Laser System, manufactured by Laser Machining, Incorporated of Somerset, Wis. These versatile machines include a indexing head having a chuck for receiving various tools, and a laser directed toward the axis of rotation of the indexing head. The indexing head is typically mounted on a table which is moveable, relative to the laser, in a plane generally perpendicular to the laser beam, so a work piece may be moved into and out of a cutting position by a computer controlling the movement of the table.

It is thus an object of the invention to provide an automated method of cutting a stent from a length of braided stock material, which creates a sphere or similar dull surface at the end of each wire of the stent.

It is also an object of the invention to provide a device for holding a length of braided stock material that does not allow the individual wire to “jump” after being cut.

It is another object of the invention to provide a device that presents the individual wires of braided stent stock to a cutting device in a predictable, repeatable, accurate manner, regardless of inconsistencies present in the braids of the stent stock.

It is further an object of the invention to provide an automated method of cutting a stent from a length of braided stock material that results in a uniform plurality of wire ends.

Another object of the invention is to provide an automated method of cutting a stent from a length of braided stock material that creates a sphere or similar dull surface at the end of each wire of the stent.

Yet another object of the invention is to provide a method of cutting a stent from a length of braided stock material is described which results in a braided stent that is resistant to unraveling.

Still another object of the present invention is to provide a method of cutting a stent from a length of braided stock, which creates a stent that provides increased comfort to the patient.

These and further objects and advantages of the present invention will become clearer in light of the following detailed description of illustrative embodiments of this invention described in connection with the drawings.

### BRIEF DESCRIPTION OF THE DRAWINGS

The illustrative embodiments may best be described by reference to the accompanying drawings where:

FIG. 1 is a series of sequential diagrams showing the formation of a sphere as a focused heat source passes through a wire or strand of meltable material at an acclivitous angle;

FIG. 2 is a perspective view of a braided stent having spherical ends, to which the present invention is directed to forming, the stent shown being greatly enlarged and loosely braided in order to show detail;

FIG. 3 is a perspective view of a preferred embodiment of the stent stock retaining device of the present invention shown in a disengaged position;

FIG. 4 is a perspective view of the retaining device of FIG. 3, shown in an engaged position;

FIG. 5 is a section view of the retaining device of the present invention, taken generally along lines 5-5 of FIG. 4;

FIG. 6 is a representation of the geometry of an individual diamond of the braided stent stock to which the present invention is directed, labeling the various dimensions of the stock in order to describe the geometry thereof mathematically;

FIG. 7 is a representation of the geometry of an end of the braided stent stock to which the present invention is directed; and,

FIG. 8 is a flowchart of the steps of a preferred embodiment of the method of the present invention.

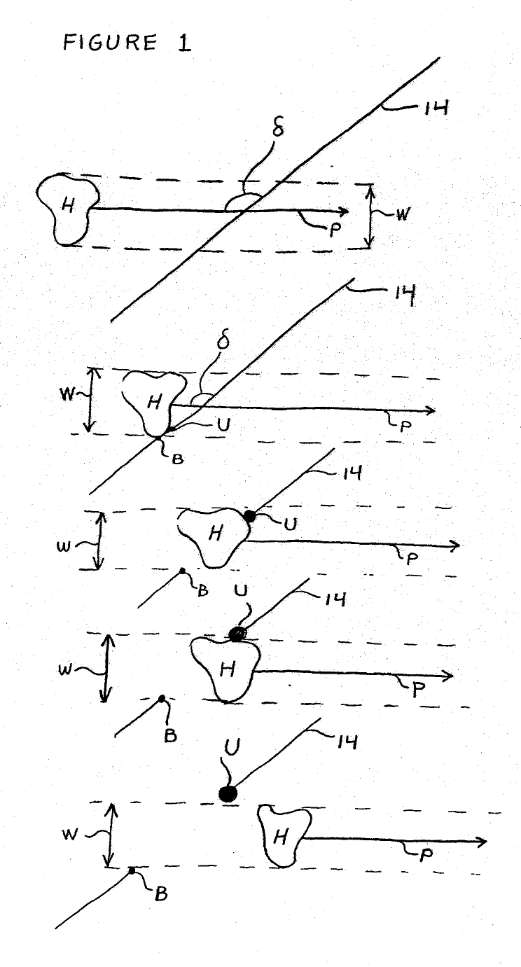

All figures are drawn for ease of explanation of the basic teachings of the preferred embodiments only. The extensions of the Figures with respect to number, position, relationship, and dimensions of the parts to form the preferred embodiments will be explained or will be within the skill of the art after the following description has been read and understood. Further, the exact dimensional proportions to conform to the specific force, weight, strength, and similar requirements will likewise be within the skill of the art after the following description has been read and understood.

Where used in the various figures of the drawings, the same numerals designate the same or similar parts. Furthermore, when the terms “top,” “bottom,” “upper,” “lower,” “first,” “second,” “front,” “rear,” “end,” “edge,” “forward,” “rearward,” “upward,” “downward,” “inward,” “outward,” “inside,” “side,” “longitudinal,” “lateral,” “horizontal,” “vertical,” “acclivitous,” “declivitous,” and similar terms are used herein, it should be understood that these terms have reference only to the structure shown in the drawings as it would appear to a person viewing the drawings and are utilized only to facilitate describing the preferred embodiments. It should be further understood that the term “sphere,” as used herein, pertains to a generally curved shape at the end of a strand and does not imply the formation of a mathematical sphere. Strands having ends which are egg shaped, tear drop shaped, generally thickened, or generally rounded are considered “spherical” as the term is used herein.

### DETAILED DESCRIPTION OF THE PREFERRED EMBODIMENTS

Device

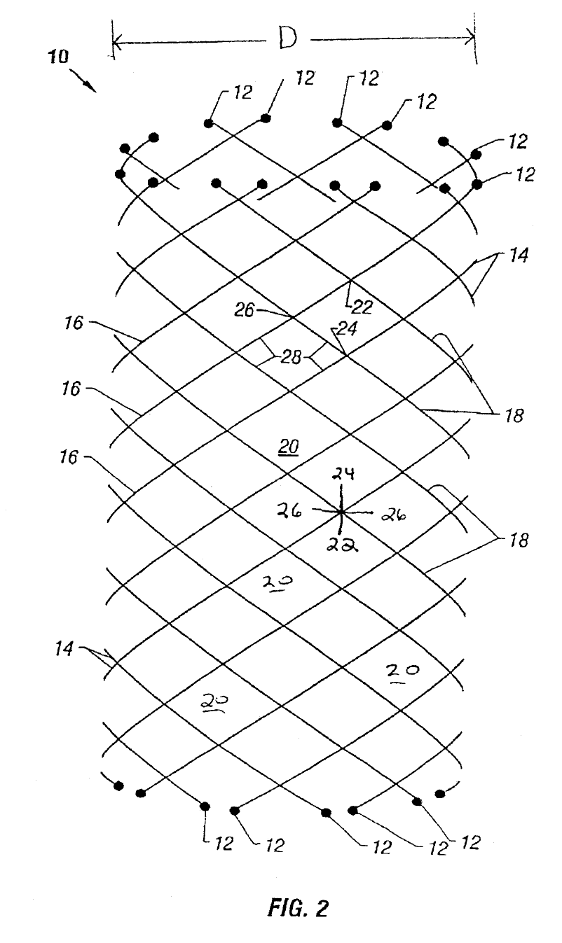

Referring now to the figures, and first to FIG. 2, there is shown a braided stent 10 to which the various embodiments of the devices and methods of the present invention are directed to forming. Stent 10 is formed such that the cut ends 12 of the wires or strands 14 are substantially spherically shaped.

Stent 10 is a segment cut from braided stent stock, which is made up of a plurality of strands 14. Strands 14 are braided such that half of the strands 14 form left-handed helixes 16 and the other half of the strands 14 form right-handed helixes 18. The various helixes 16 and 18 are alternately woven together to define a plurality of diamond-shaped openings 20. Openings 20 have upper apexes 22, lower apexes 24 and side apexes 26, which are formed by the intersections of the individual strands 14. The strand lengths between the intersections define the sides 28 of the diamonds 20. It is readily apparent from the figure that any given intersection of two strands or wires 14 serves as common point for four diamonds 20 by being the upper apex 22 for one, the lower apex 24 for another, and side apexes 26 for the other two openings 20. It should be noted that FIG. 2 shows only the upper hemisphere of a stent 10 in detail in order to preserve clarity of representation.

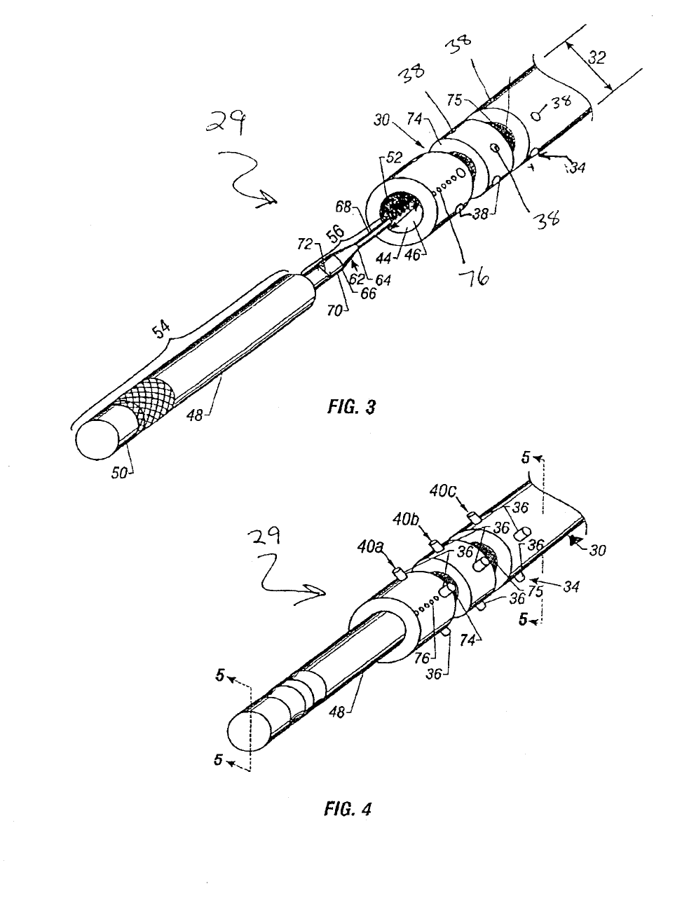

Referring now to FIGS. 3-5, there is shown a device 29 for facilitating the controlled handling of a length of stent stock while the stock is cut to a predetermined length in order to form a stent 10. Device 29 preferably includes a mandrel 30. Mandrel 30 has an outside diameter 32 sized to receive a given size of stent stock. Diameter 32 should be slightly smaller than the inside diameter of the corresponding stent stock, measured while the stock is in a relaxed condition, so that the stock slides easily over mandrel 30 and so no internal stresses are created within the stock due to it being placed on mandrel 30. Mandrel 30 includes an anchoring mechanism 34 for temporarily fixing or securing a length of stent stock to mandrel 30 in such a manner that the exact location of the various intersections of strands 14 can be positioned to avoid the path of a cutting device. It is also preferable that no significant internal stresses are imparted into the strands 14.

The envisioned embodiment of anchoring mechanism 34 shown in FIGS. 3-5 includes a plurality of pins or protuberances 36 slideably housed within a plurality of apertures 38 defined by mandrel 30. Preferably, pins 36 and apertures 38 are arranged in longitudinally spaced apart sets 40. The Figures depict an embodiment having three sets, 40a, 40b and 40c. Using three sets 40 has been found to be best suited when two cutting positions are desired, as will be discussed in more detail below. However, it is understood that if only one cutting position is needed, two sets 40 are optimal. It has been found advantageous to provide one set 40 on either side of each cutting position. This configuration ensures that stent stock on either side of a cut will be secure.

Similarly, the figures show four pins 36 and four apertures 38 per set, angularly spaced ninety degrees apart from each adjacent pin 36 and aperture 38. This configuration facilitates ease of manufacturing in that two opposite apertures 38 may be drilled or machined with one tool stroke. For purposes of securing stent stock to mandrel 30, three or five pins 36 per set 40 would also be effective.

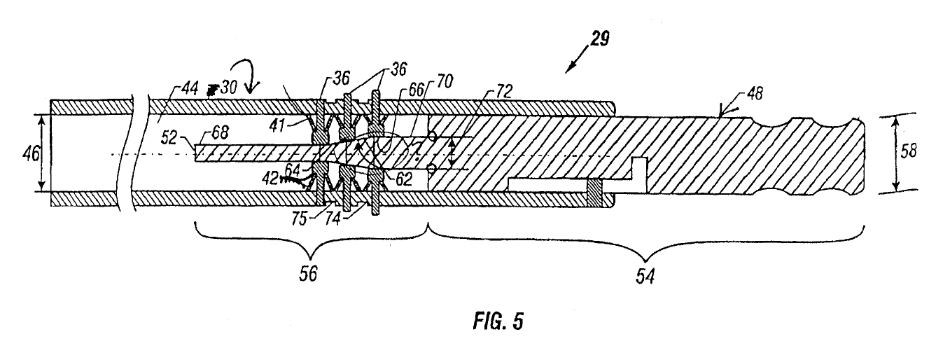

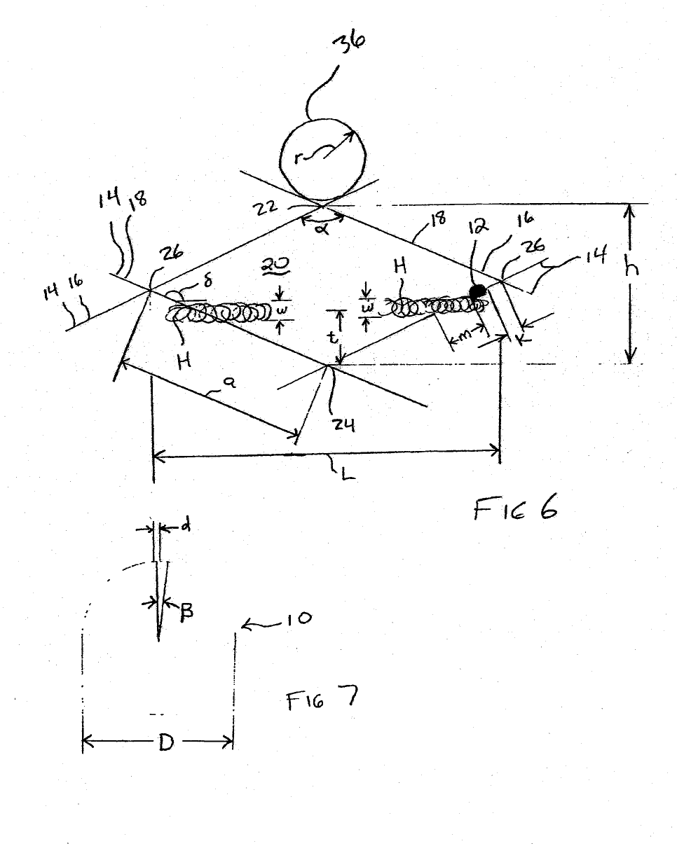

Ease of stent stock removal and readjustment is achieved by biasing pins 36 inwardly. As best seen in FIG. 5, coil springs 41 surround each pin 36 and act against mandrel 30 and against a pin flange 42 defined by each pin 36, thereby urging each pin 36 inwardly. Preferably, spring 41 is attached at one end to mandrel 30 and at an opposite end to flange 42, thereby preventing pins 36 from becoming unseated within apertures 38.

Apertures 38 lead into an inner channel 44 defined by mandrel 30 and preferably concentric therewith. Inner channel 44 is characterized by an inner diameter 46 which is small enough to provide the appropriate thickness between outer diameter 32 and inner diameter 46 of mandrel 30 such that pins 36 are adequately supported and long enough to protrude through the diamond shaped holes or openings 20 of the stent stock. Though any appropriate mechanism for causing pins 36 to protrude from apertures 38 would be acceptable, it is envisioned that an activation dowel 48 is provided. Activation dowel 48 preferably includes a tip 52, a handle portion 54 and an activation portion 56. Handle portion 54 has an outside diameter 58 sized to fit within inner channel 44 of mandrel 30. Preferably, outer diameter 58 is only slightly smaller than inner diameter 46 such that a snug fit is provided. Handle portion 54 is of sufficient length that stability is provided to activation dowel 48 when inserted within inner channel 44. Handle portion 54 is also preferably of sufficient length that when activation dowel 48 is fully inserted within inner channel 44 of mandrel 30 a segment of handle portion 54 remains outside of mandrel 30 such that it may be grasped for removal.

Activation portion 56 is adjacent handle portion 54 and includes an angled portion 62 having a front 64 and a rear 66. Front 64 has a smaller outside diameter than does rear 66. In the preferred embodiment, activation dowel 48 also includes a first cylindrical segment 68 which extends from tip 52 to angled portion front 64 and a second cylindrical segment 70 extending from angled portion rear 66 to handle portion 54. First cylindrical segment 68 functions to initially align activation dowel 48 when inserted into inner channel 44 of mandrel 30. This can best be seen in FIG. 5. Second cylindrical segment 70 has an outer diameter 72 which is sized to cause pins 36 to fully protrude from mandrel 30 when activation dowel 48 is fully inserted. It can also be seen that the difference between outer diameter 72 and inner diameter 46 is great enough to allow sufficient space between second cylindrical segment 70 and mandrel 30 to contain pin 36 and spring 41 in a compressed state.

In addition to apertures 38, it is preferable that mandrel 30 further comprise at least one cutting groove or slot 74 for preventing damage to mandrel 30 during a cutting operation. Cutting groove 74 provides a space between the outer surface of mandrel 30 and the strands of stent stock intended to be cut. Preferably, a first slot 74 is provided between pin sets 40a and 40b, and a second slot 75 is provided between pin sets 40b and 40c.

Optionally, mandrel 30 may also include a plurality of reference markings 76 to aid in the proper placement of a length of stent stock in determining the resulting length of cut stent 10. Markings 76 are preferably spaced apart by a distance approximately equal to the distance between an upper apex 22 and a lower apex 24 of any given diamond opening 20 of the stent stock for which device 29 is designed. Markings 76 are also preferably aligned longitudinally as seen in FIGS. 3-4.

Mathematical Relationships

The physical preferred embodiments having thus been described it is now important to define the mathematical relationships between the various measurements of the given stent stock and the physical locations and sizes of the pins 36 and grooves 74 of mandrel 30. Reference is made to FIGS. 6 and 7.

Pins 36 are preferably sized to have a radius r that snugly fits within any given diamond shaped opening 20 of the stent stock. It can be shown that the largest pin radius r which can fit within a diamond can be represented mathematically by the formula:

$$r = \frac{Lh}{2 {( {h}^{2} + {L}^{2} )}^{\frac{1}{2}}}$$

where h represents the inside height of diamond opening 20 measured from its lower apex 24 to its upper apex 22 and L represents the inside length of diamond opening 20 as measured from one side apex 26 to an opposite side apex 26.

If r represents the largest possible pin radius which can fit within a diamond of height h and length L, then it can be shown that:

$$lim r = \frac{h}{2}$$

It should be noted that, for purposes of mathematical representation and ease of calculations, some of the formulas presented herein make the assumption that diamonds 20 and strands 14 lie in a flat plane. In reality, stent 10 is cylindrical and diamonds 20 and wires 14 necessarily follow the curve of stent 10. However, it has been found that, in practice, making the mathematical assumption that the diamonds 20 and strands 14 lie in a flat plane, has not affected the desired results and that the incremental differences between the assumed flat plane and the actual cylindrical surface are relatively inconsequential.

It is important to provide a heat source, preferably a laser beam, having an effective cutting area small enough to cut strands 14 while avoiding intersections of left-hand helixes 16 and right-hand helixes 18. In order to determine the appropriate position of a heat or melting source H emanating an energy field having an effective width w, it is necessary to define and determine the relationships between the heat source width w, the length a of any given side of diamond 20, the length m which represents the length of the strand which will be melted by the heat source H, the angle α which is the inner angle between the strands of the upper apex 22 or lower apex 24, and the outer diameter D of the stent stock. These variables having been defined, it can be shown that

$$m = \frac{w}{cos ( \frac{α}{2} )} and , \\ \frac{m}{a} = 7.6 ( \frac{w}{D} ) tan ( \frac{α}{12} )$$

where m/a represents the portion of wire material of a given side of a diamond 20 which will be melted and displaced by heat source H to form a gap and a sphere S.

Having established these relationships, an appropriate axial separation between the center of the cutting path of the heat source H and the upper apex 22 or lower apex 24 of a given diamond 20 can be determined. Referring to FIGS. 6 and 7, this distance is represented by t. It can be seen that t may fall within a range of values. The range varies, depending on the desired sphere S size, the width w of the heat source, and the desired strand length k between the sphere S and the intersection of the strands 14.

In a preferred embodiment of the present invention it is desired to cut alternating strands to more predictably form significant spheres S on one side of a stent, and also to protect the mandrel from damage due to repeated exposure to the heat source H. This can be accomplished by turning the heat source H on while cutting and turning the heat source H off while the stent is being rotated to the next cutting position. More preferably, when the heat source H is a laser beam, the beam may be alternately directed toward and away from the strand by using a reflector or by blocking and unblocking the beam using a shutter. In order to determine the appropriate timing of the activation and deactivation of heat source H, it is necessary to determine the angle β of stent rotation during which a heat source should be turned on. In other words, an angle β needs to be defined, which represents the angular length of the melted portion m of wire arm a. Angle β may be represented by the following formula:

$$β = 114.6 ° ( \frac{w}{D} ) tan ( \frac{α}{2} )$$

This formula holds true for stent stock having twelve left-hand helixes 16 interwoven with twelve right hand helixes 18 for a total of twenty-four strands 14. Furthermore, it has been found that in order to create spheres S on the same side of the heat source H, the left-hand helix 16 should be cut during one stent rotation direction while the right-hand helix 18 should be cut while the stent stock is rotating in an opposite direction. The preferred method of forming spheres S on the ends of the strands 14 will be discussed further below. But having this in mind, the angle during which heat source H should be deactivated, defined herein as γ, is related to β in the following manner:

$$γ = \frac{360 ° - 12 β}{12}$$

An example of a preferred embodiment is now provided. Given stent stock having an outside diameter D of 14 millimeters, strand diameters of 0.17 millimeters and braid angle α of 145 degrees, favorable results have been obtained using a mandrel having an outer diameter 32 on the order of 13.7 millimeters and defining an inner channel 44 with an inner diameter 46 on the order of 9.53 millimeters, more preferably 9.53+−0.03 millimeters. Pins 36 preferably have a radius r of 0.5 millimeters. Furthermore, pertaining to activation dowel 48, outer diameter 72 of second cylindrical segment 70, is on the order of 4.01+−0.05 millimeters, while outer diameter 58 of handle portion 54 is on the order of 9.37−9.50 millimeters to fit nicely within inner channel 44.

It should be noted that the acclivitous angle δ at which a strand 14 relatively approaches oncoming heat source H was not necessary for purposes of explaining the above mathematical relationships. However, it is related to angle α in the following manner:

$$δ = \frac{α}{2} + 90 °$$

δ is preferably between 130 and 175 and more preferably on the order of 162, for best results.

Method

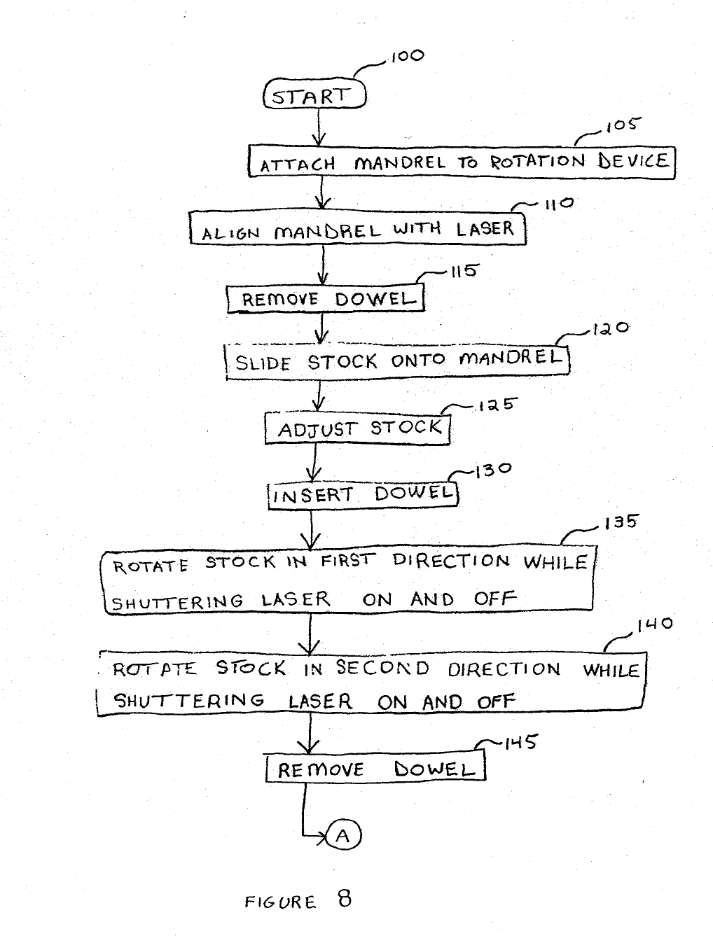

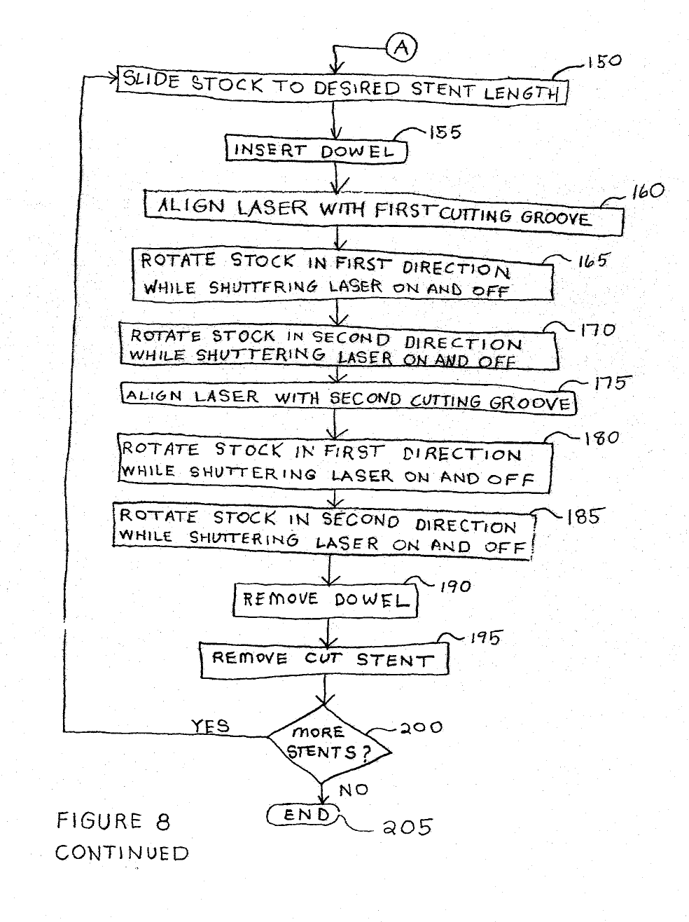

The physical embodiments and mathematical relationships having thus been described, attention can now be drawn to FIG. 8, a flow chart detailing the preferred steps of the method of the present invention. The process starts at step 100. Here, the assumption is made that stent stock is being used that has not yet been cut to form spheres S on one end. In the event that the stent stock already has spheres S formed on one end, the method of the present invention should start at step 150.

First, the mandrel 30 is attached to a rotation device, preferably the indexing head of a laser cutting machine, at 105 in preparation for cutting. It is understood that mandrel 30 could already have been attached to the indexing head and that certain steps of the sequence described herein could be rearranged as would be seen by one skilled in the art. The mandrel is then positioned below the laser of the laser cutting machine so that the beam is aimed at second cutting groove 75 at 110. This is preferably accomplished by moving the indexing head relative to the laser after mandrel 30 has been placed within the chuck of the indexing head. It has been found that it is preferable to use the center of one of the pins 36 as a target when aligning the mandrel 30 under the laser. The laser can then be offset from the pin 36 to the second cutting groove 75 by entering the known distance between the groove 75 and the pin 36 into a computer controlling the movement of the table on which the indexing head is mounted. This is preferable because the pin 36 provides a more precise point on the mandrel 30. The groove 75 is a relatively wide area designed just to protect the mandrel 30 against over exposure to the heat source H.

The activation dowel 48 is then removed from the mandrel 30 at 115. Once the activation dowel 48 has been removed, the stent stock is slid onto the mandrel 30 at 120. The stent stock is then adjusted axially along the length of the mandrel 30 at 125 such that all three pin sets 40 are able to engage diamonds 20. The activation dowel 48 is then inserted at 130. The dowel 48 is inserted slowly such that the first pin set 40a protrudes first and finds diamond openings 20 in the stent stock. The subsequent pin sets 40b and 40c then protrude sequentially, also finding diamond openings 20 in the stent stock.

At 135, the mandrel 30 and the stent stock are rotated in a first direction, at least one revolution, preferably at a speed of less than 10 revolutions per minute, more preferably on the order of 6 revolutions per minute. While the mandrel 30 is rotating in this first direction, the laser beam is shuttered on and off. The shuttering of the laser is timed such that the laser is shuttered on and cutting whenever an acclivitous strand 14 is below the laser. Once a strand 14 has angularly passed completely beyond the laser beam, the laser is shuttered off until another acclivitous strand 14 is presented. This will result in the laser being shuttered on twelve times during one revolution. At 140, after the laser has rotated at least one revolution in a first direction and all acclivitous strands 14 have been cut and spheres S formed thereon, the mandrel 30 is rotated in a second, opposite direction in order that the remaining strands 14 may present acclivitous angles relative to the laser beam. While the mandrel 30 is rotating in the second direction, the laser is again shuttered on and off, cutting and forming spheres S on the remaining strands 14. The laser is preferably shuttered off whenever a strand 14 is not present to avoid unnecessary heating of mandrel 30, declivitous strands 14, and any spheres S that were formed during the first rotation. The activation dowel 48 is then removed from mandrel 30 at 145, thereby allowing springs 41 to urge pins 36 inwardly, disengaging pins 36 from the stent stock.

At this point, the stent stock has been given an end that is complete with spheres S on the ends of each of the wires 14. This end may then be used to form the end of a cut stent 10. At 150, it is necessary to slide the stent stock along the length of the mandrel 30 an appropriate distance such that when cutting takes place along the first cutting groove 74, a stent 10 of a desired length results with spheres S formed at both ends. It is understood, however, that it may be desirable to form a length of stent stock with spheres S on only one end and that the method herein described may be easily modified to do so. Reference markings 76 may aid in sliding the stent stock the appropriate distance to form a stent of a desired length. Once the stock is in a desired position, the activation dowel 48 is reinserted within mandrel inner channel 44 at 155. While the activation dowel 48 is being inserted into inner channel 44, thereby causing pins 36 to protrude from apertures 38, it may be desired to adjust the position of the stent stock on mandrel 30 so pins 36 do not encounter any interference with the intersections of strands 14. Care should be taken while adjusting the stent stock such that an undesired length is not achieved.

The mandrel is then moved under the laser so that the first cutting groove 74 is aligned under the laser at 160. This is preferably accomplished by entering an appropriate command into the computer which then moves the table on which the indexing head is mounted, obviating the need to retarget the laser at a pin 36. Again, the mandrel is rotated in a first direction at 165 and the laser is appropriately shuttered on and off to cut acclivitous strands 14. After at least one revolution is completed, the stock and mandrel 30 are then rotated in a second direction at 170 while the laser is again shuttered on and off to cut remaining strands 14.

One complete stent 10 has now been cut. However, in a preferred embodiment, two cutting grooves 74 and 75 are provided such that spheres S may be formed using second cutting groove 75 on the newly cut end of the stent stock without having to move the stent stock along the length of mandrel 30. Therefore, at 175, the mandrel is aligned so that second groove 75 is beneath the laser. As the relative position of the mandrel and the laser has already been established, it is not necessary to target the laser to a pin 36, rather, the computer may be used to move the table an appropriate distance to align the second groove 75 below the laser. The stent stock is then again rotated in a first direction, not necessarily the same direction as the first direction of the first cut, at least one revolution at 180; and while this is happening, the laser is again shuttered on and off to cut acclivitous strands 14 in this first direction. Again, at 185, the stock is rotated in a second direction while the laser is shuttered on and off to cut remaining strands 14.

At this point, on mandrel 30, there exists a cut stent 10, a piece of scrap stent stock having no spheres S on the strands 14 of either end, and a length of stent stock having spheres S formed on the ends of the wires 14 making up the stent stock. Activation dowel 48 is then removed at 190, and the cut stent 10 is slid off the mandrel 30, along with the scrap, at 195.

At 200, a decision is made as to whether more stents 10 are desired to be cut from this length of stent stock. If more stents 10 are desired, the process is repeated starting at step 150. If no further stents 10 are desired, either because the desired number of stents 10 have been formed or because there is not enough remaining length of the stent stock to form another stent 10, the process is finished at 205.

Results

It has been found that by practicing the preferred embodiments of the present invention, namely, using the structures taught herein and following the above method to acquire the disclosed mathematical relationships, stents can be formed with ends having spheres S that are uniquely uniform in size and shape. Moreover, an extremely predictable length of braid material is melted to form the spheres S and a desired resulting stent length can be achieved with surprising consistency.

For example, when cutting a length of braided stent stock made of braids having diameters of 0.17 millimeters and helixes which present acclivitous angles δ on the order of 162.5 degrees to a laser found on an Eagle 500 CO₂ Laser System, at an angular speed of 6 rotations per minute, in accordance with the preferred embodiments of the present invention, a repeatable sphere S size of 0.012-0.013 millimeters can be attained.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. For example, it would be foreseeable, using the teachings of the present invention, to create a similar device having more pins and grooves such that two lasers could be used simultaneously, one cutting each end of a stent. It is also foreseeable, and within the envisioned embodiments, to utilize a laser system or other heat source which moves the laser beam while keeping the planar position of the indexing head fixed. A third example of an alternate specific form is using multiple passes of a heat source across a predetermined length of wire to create effective energy field width w, as opposed to using a single pass, to form a sphere thereon. This may be desired when using an energy field having an extremely small effective heating area. These are merely three examples of other specific forms in which the present invention may be embodied. Accordingly, the present invention is not limited in the particular embodiments, which have been described in detail herein. Rather, reference should be made to the appended claims as indicative of the scope and content of the present invention.

What is claimed:

1. A stent for placement in a body lumen comprising:
a plurality of right-handed helical strands, each having a first end and a second end;
a plurality of left-handed helical strands, each having a first end and a second end;
said plurality of right-handed helical strands being interwoven with said plurality of left-handed helical strands such that said stent has a periphery defined by a series of diamond-shaped openings; and,
said first end of each of said plurality of right and left-handed helical strands including a sphere formed from melting a predetermined length of each of said first ends, said predetermined length being partially defined by an acclivitous angle at which each of said first ends contact an energy field emanating from a melting source.

2. A stent according to claim 1, wherein said second end of each of said plurality of right and left-handed helical strands includes a sphere formed from melting a predetermined length of each of said second ends, said predetermined length being partially defined by an acclivitous angle at which each of said second ends contact an energy field emanating from a melting source.

3. A stent according to claim 1, wherein said predetermined length is further defined by a speed at which each of said first ends and said energy field pass each other.

4. A stent according to claim 1, wherein said melting source is a laser.

5. A stent according to claim 1, wherein said acclivitous angle is in the range of approximately 130 to 175 degrees.

6. A stent according to claim 1, wherein said predetermined length is further defined by an effective path width of said energy field emanating from said melting source.

7. A stent according to claim 3, wherein said speed is a rotational speed.

8. A stent according to claim 7, wherein said rotational speed is less than 10 rotations per minute.

9. A stent according to claim 8, wherein said rotational speed is on the order of 6 rotations per minute.

10. A stent according to claim 2, wherein said melting source used to form said spheres on said first ends is the same as said melting source used to from said spheres on said second ends.
